(19) United States
(12) Patent Application Publication
(10) Pub. No.: US 2013/0180021 A1
(43) Pub. Date: Jul. 18, 2013

(54) FABRIC SUNSHADE ACCESSORY FOR GLASSES

(71) Applicant: JOHN ADAM DANNER, Tampa, FL (US)

(72) Inventor: JOHN ADAM DANNER, Tampa, FL (US)

(21) Appl. No.: 13/348,867

(22) Filed: Jan. 12, 2012

(51) Int. Cl. A42B 1/06; A61F 9/00

(52) U.S. Cl. 2/10; 2/172

(57) ABSTRACT

A fabric sunshade can be assembled with glasses having temples. The sunshade is made of a piece of fabric and is provided with at least one passageway having a pair of open ends. The passageway is located adjacent to or near an edge of the sunshade for receiving the temples of the glasses to attach the sunshade to the glasses. When the glasses and sunshade are worn by a person a portion of the passageway is disposed on a back side of the head of the person and the sunshade extends downward to shade at least a portion of a neck of the person.

### FIELD OF THE INVENTION

The present invention relates to a fabric sunshade for assembly with glasses to shield a portion of a wearer from the sun.

### BACKGROUND OF THE INVENTION

Persons exposed to sunlight should attempt to reduce the exposure of skin on the back of the neck and ears to ultraviolet rays that may cause dry skin or, in extreme cases, skin cancer. Often persons exposed to sunlight employ glasses with darkened lenses to aid in protecting their eyes from ultraviolet rays. Persons have addressed these problems in the past by wearing hats with broad brims or wearing hats provided with fabric sunshades. Many persons do not like to wear hats, or prefer stocking style hats or to tie a bandana on their head. These persons however still need to protect their neck and ears from ultraviolet rays, and a means for doing this is provided by the present invention which entails a fabric sunshade that may be secured to a person's head using glasses.

### DISCUSSION OF THE PRIOR ART

One way to protect eyes from direct sunlight is a simple sun visor such as that taught in U.S. Pat. No. 2,629,869. Another way to shade the ears and neck of a person is by wearing a hat with a large brim that is at least semi-rigid such as that taught in U.S. Pat. No. 4,771,477.

Another way to protect a person's ears and neck from direct sunlight is to wear a cap or hat that is provided with a fabric sunshade attached to or integral with the cap or hat as taught for example in: U.S. Pat. No. 2,897,510; U.S. Pat. No. 5,081,717; U.S. Pat. No. 5,083,317; U.S. Pat. No. 5,400,4440; U.S. Pat. No. 5,694,647; U.S. Pat. No. 5,153,043; U.S. Pat. No. 5,161,259; U.S. Pat. No. 5,355,535; U.S. Pat. No. 5,448,778; U.S. Pat. No. 5,669,075; U.S. Pat. No. 5,701,609; U.S. Pat. No. 6,966,072; US 2002/0035745 A1; U.S. D 474,327; and the “Universal Sun Shade” disclosed at http://www.amazon.com/Dr-Shade-Universal-Sun/dp/B0045E5XE0.

Another way to shade the ears and neck of a person is by wearing a headband that extends completely around the head with a fabric sunshade fixed directly to the headband as taught in U.S. Pat. No. 5,046,195; U.S. Pat. No. 5,323,491; US 2006/0212995 A1; and the SolFlap™ head wrap as advertised at Solflaps.com on the internet, http://www.solflaps.com/asi.html.

U.S. Pat. No. 6,032,292 discloses a bandanna or scarf formed with holding elements thereon, and may include a number of flexible layers which provide protection from the elements. A top portion of the bandanna or scarf may include elastic elements to aid in securing the bandanna or scarf in place on the nose of a wearer, or may have a pair of drawstrings with locking elements to enable the bandanna or scarf to be adjustably tightened when wearing. The drawstrings may also include loops for supporting glasses thereon. However, the glasses are not used to support or maintain the bandanna or scarf in place on a wearer.

U.S. Pat. No. 4,821,341 discloses a size adjustable sun-visor and headpiece combination adapted to be worn on the head of a wearer, the headpiece having a front portion to run across the forehead of the wearer and a rear portion extending downwardly to cover the back of the neck of the wearer. An elongated headband is secured to opposite sides of the rear portion of the headpiece. A button is provided on each of the right side and left side portions of the headband to secure the sun-visor to the headband, the sun-visor including two elastic straps secured to opposite sides thereof to form end loops to accommodate associated ones of the buttons to attach the sun-visor to the headband while permitting the end loops to be detached from their respective buttons for removal of the sun-visor from said headband. The sun-visor may be raised or lowered at the discretion of a wearer of the device. This device is not designed for use with ordinary glasses, but rather requires a sun-visor having a specific configuration.

### SUMMARY OF THE INVENTION

There is provided in accordance with the present invention a fabric sunshade that can be assembled with glasses having temples. The sunshade comprises a piece of fabric and provided with at least one passageway having a pair of open ends. The passageway is located adjacent to or near an edge of the sunshade for receiving the temples of the glasses to attach the sunshade to the glasses. When the glasses and sunshade are worn by a person a portion of the passageway is disposed on a back side of the head of the person and the sunshade extends downward to shade at least a portion of a neck of the person.

### BRIEF DESCRIPTION OF THE DRAWINGS

FIG. 1 is a perspective view of a first embodiment of a fabric sunshade assembled with a pair of glasses.

FIG. 2 is a top view looking down on the first embodiment of the fabric sunshade assembled with a pair of glasses.

FIG. 3 is a front plan view of the first embodiment of the fabric sunshade for use with glasses.

FIG. 4 is a rear plan view of the first embodiment of the fabric sunshade for use with glasses.

FIG. 5 is a plan view of the first embodiment of the fabric sunshade as a blank during the process of manufacturing the sunshade.

FIG. 6 is a schematic view of the first embodiment of the fabric sunshade from the side during the process of manufacturing the sunshade.

FIG. 7 is a schematic side view of the first embodiment of the completed sunshade.

FIG. 8 is a perspective view of a second embodiment of a fabric sunshade assembled with a pair of glasses.

FIG. 9 is a front plan view of the second embodiment of a fabric sunshade.

FIG. 10 is an exploded view of a third embodiment of a fabric sunshade for use with glasses.

FIG. 11 is a schematic side view of the third embodiment of the fabric sunshade.

FIG. 12 is a front plan view of the third embodiment of the fabric sunshade.

FIG. 13 is a perspective view of a fourth embodiment of a fabric sunshade assembled with a pair of glasses.

FIG. 14 is a top view looking down on the fourth embodiment of the fabric sunshade assembled with a pair of glasses.

FIG. 15 is a front plan view of the fourth embodiment of the fabric sunshade for use with glasses.

FIG. 16 is a plan view of the fourth embodiment of the fabric sunshade as a blank during the process of manufacturing the sunshade.

FIG. 17 is an exploded view of a fifth embodiment of a fabric sunshade for use with glasses.

FIG. 18 is a schematic side view of the fifth embodiment of the fabric sunshade.

FIG. 19 is a front plan view of the fifth embodiment of the fabric sunshade.

### DETAILED DESCRIPTION OF THE INVENTION

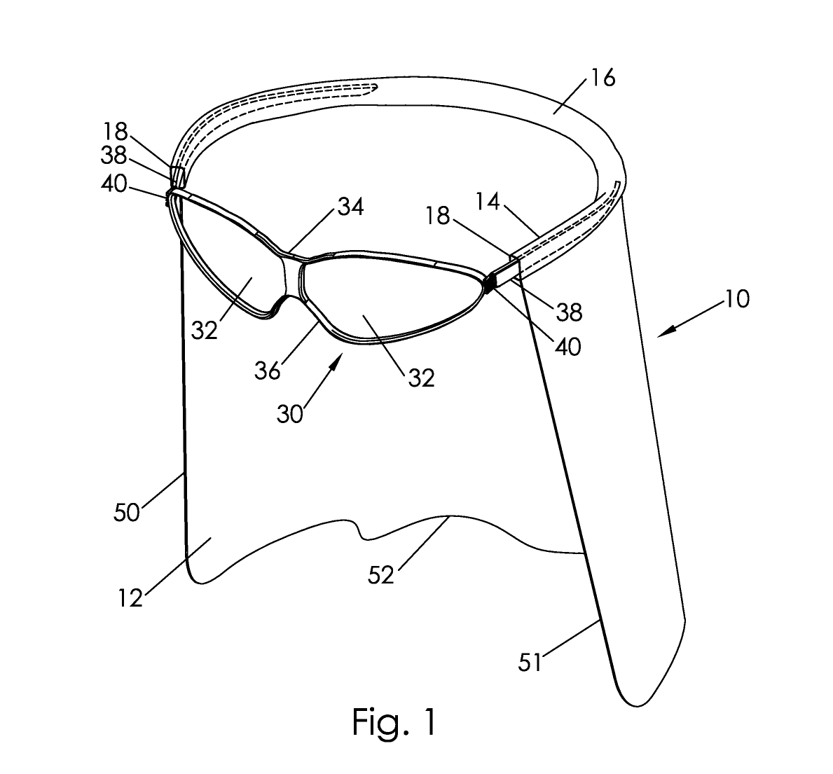

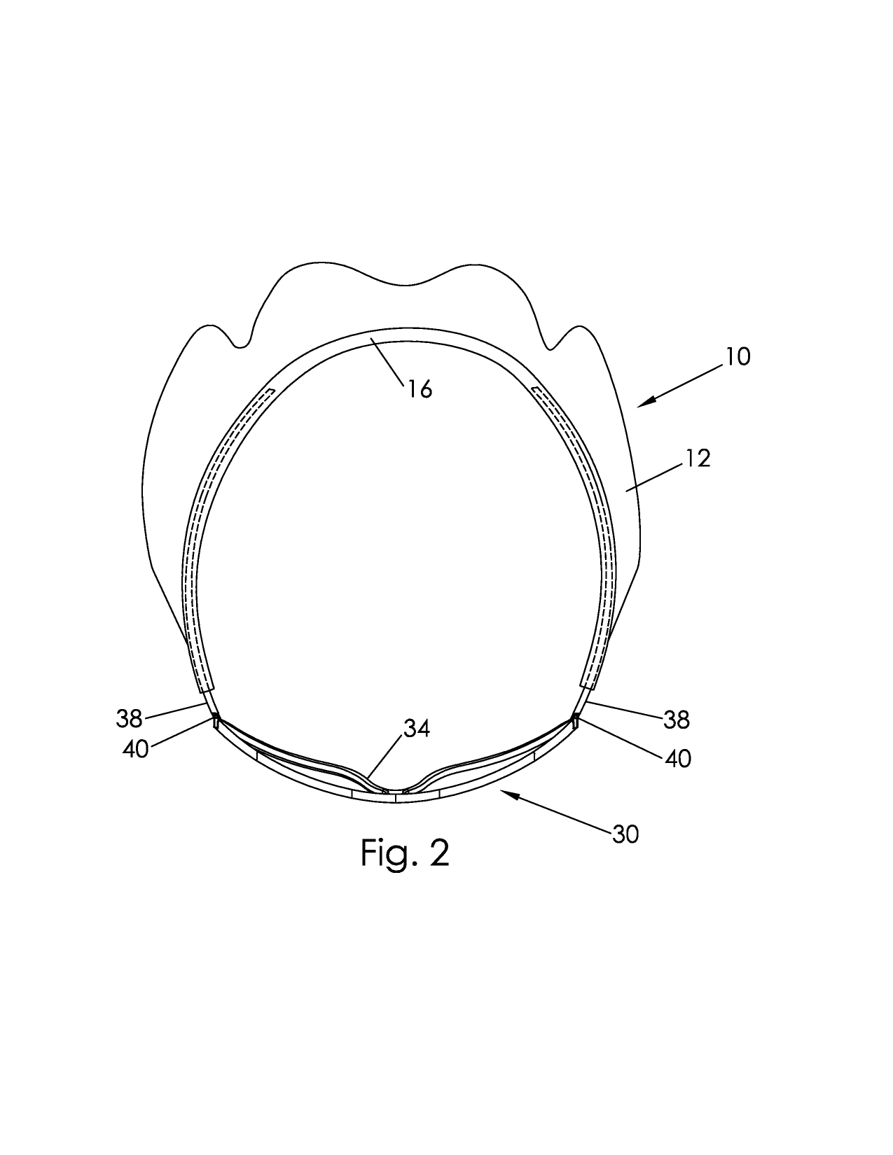

Referring first to FIGS. 1 and 2 there is shown a perspective view of a first embodiment of a fabric sunshade 10 assembled with a pair of glasses 30 and a top view looking down on the same assembly. As used herein an in the claims the terms “glasses” and “pair of glasses” are understood to have their widely accepted meanings of a device to compensate for defective vision or to protect the eyes from light, dust, and the like, consisting usually of two glass or plastic lenses 32 set in a frame 34 that includes a nosepiece 36 called a bridge for resting on the bridge of the nose and two sidepieces 38 called temples that extend over and/or behind the ears to help hold the frame in place. Hinges 40 connect the front part of the frame to the temples and allows the temples to swing. As used herein and in the claims the term sunshade is understood to have the widely accepted meaning of an article that is used or worn to protect a person from the sun's rays, including at least a portion of the ultraviolet rays that art a component of sunlight. A sunshade of the present invention may be assembled with glasses having clear lenses, tinted lenses, lenses that darken when exposed to sunlight, corrective lenses, non-corrective lenses, or lenses having any combination of these features.

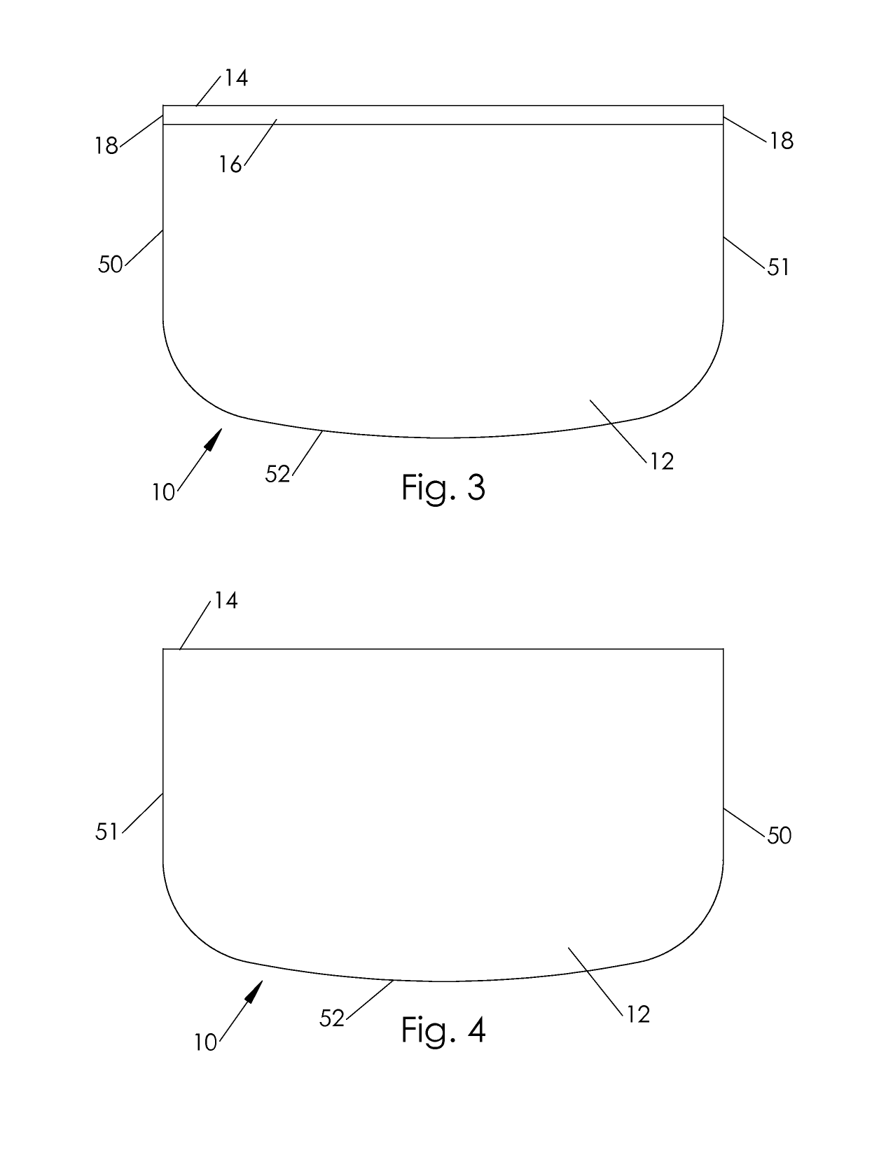

FIG. 3 is a front plan view and FIG. 4 is a rear plan view of the first embodiment of the fabric sunshade 10 for use with glasses. The sunshade comprises a piece of fabric 12, the sunshade has an edge 14 and is provided with at least one passageway 16 located near the edge 14 of the sunshade and extends at least substantially parallel to the edge of the sunshade. The passageway 16 has a pair of open ends 18 for receiving the temples 38 of the glasses to attach the sunshade to the glasses. As shown in FIGS. 1 and 2 sunshade extends between the temples of the glasses and when the glasses and sunshade are worn by a person the passageway extends along a back side of the head of a wearer and the sunshade extends downward to shade at least a portion of a neck of the wearer. As shown a sunshade of the present invention is designed to protect both the ears and at least a portion of the neck of a person wearing an assembly of the sunshade and glasses.

While it is contemplated that a sunshade according to each of the embodiments disclosed herein may be manufactured using any suitable fabric, whether or not it is stretchable, it is believed to be advantageous for the fabric employed in the piece of fabric of each of the embodiments disclosed herein to comprise a four-way stretch fabric. As used herein and in the claims the term four-way stretch fabric is understood to have its' widely accepted meaning of a fabric, such as spandex, that stretches in both directions, crosswise and lengthwise. Spandex is a synthetic fiber composed of a long-chain polymer used in the manufacture of garments to add elasticity. Four way stretch fabric is widely available, distributors include for example: Pine Crest Fabrics, 6015 NE 80th Avenue, Portland, Oreg. 97218, which is marketed at http://www.pinecrestfabrics.com/category/show/2; and Sportek International, Inc., 920 Gladys Avenue, Los Angeles, Calif. 90021, which is marketed at http://sportek.com/cgi-bin/index.cgi?cart_id=1324249125.31647&pid=2&back=0&category=Nylon_Spandex_Solids. It is also advantageous for the fabric employed in the piece of fabric of each of the embodiments disclosed herein to comprise a fabric designed for sun protection and rated for its level of ultraviolet (UV) protection. A special weave structure and denier (related to thread count per inch) may produce sun protective properties. In addition, some textiles and fabrics employed in the use of sun protective clothing may be pre-treated with UV inhibiting ingredients during manufacture to enhance their UV blocking capacity. An Ultraviolet Protection Factor (or “UPF”) rating system measures the UV protection provided by fabric. It is very similar to the SPF rating system used for sunscreens. A garment with a UPF of 50 only allows 1/50th of the UV radiation falling on the surface of the garment to pass through it. In other words, it blocks 49/50ths or 98% of the UV radiation. Fabrics that have both four-way stretch and are designed for sun protection are commercially available, distributors include for example: Pine Crest Fabrics, 6015 NE 80th Avenue, Portland, Oreg. 97218, which is marketed at http://www.pinecrestfabrics.com/category/show/2; and Sportek International, Inc., 920 Gladys Avenue, Los Angeles, Calif. 90021, http://sportek.com/cgi-bin/index.cgi?cart_id=1324249125.31647&pid=2&back=0&category=Nylon_Spandex_Solids.

Referring again to FIGS. 3 and 4 a sunshade of each of the embodiments disclosed herein has a long edge 14 that is straight, and is located at the top of the sunshade when the sunshade is assembled with glasses. A pair of side edges 50, 51 each extends from the top edge 14 and extend substantially downward when the sunshade is assembled with glasses. A bottom edge 52 of the sunshade is preferably curvilinear and merges with the side edges 50, 51 following a curved path. The sunshade may have any dimensions desired to accommodate wearers of various sizes, but it is believed that a single size of sunshade may accommodate a majority of adults because of the stretch characteristics of the four-way stretch fabric. Elastic characteristics of the four-way stretch fabric aids in retaining the glasses in a proper position on a wearer.

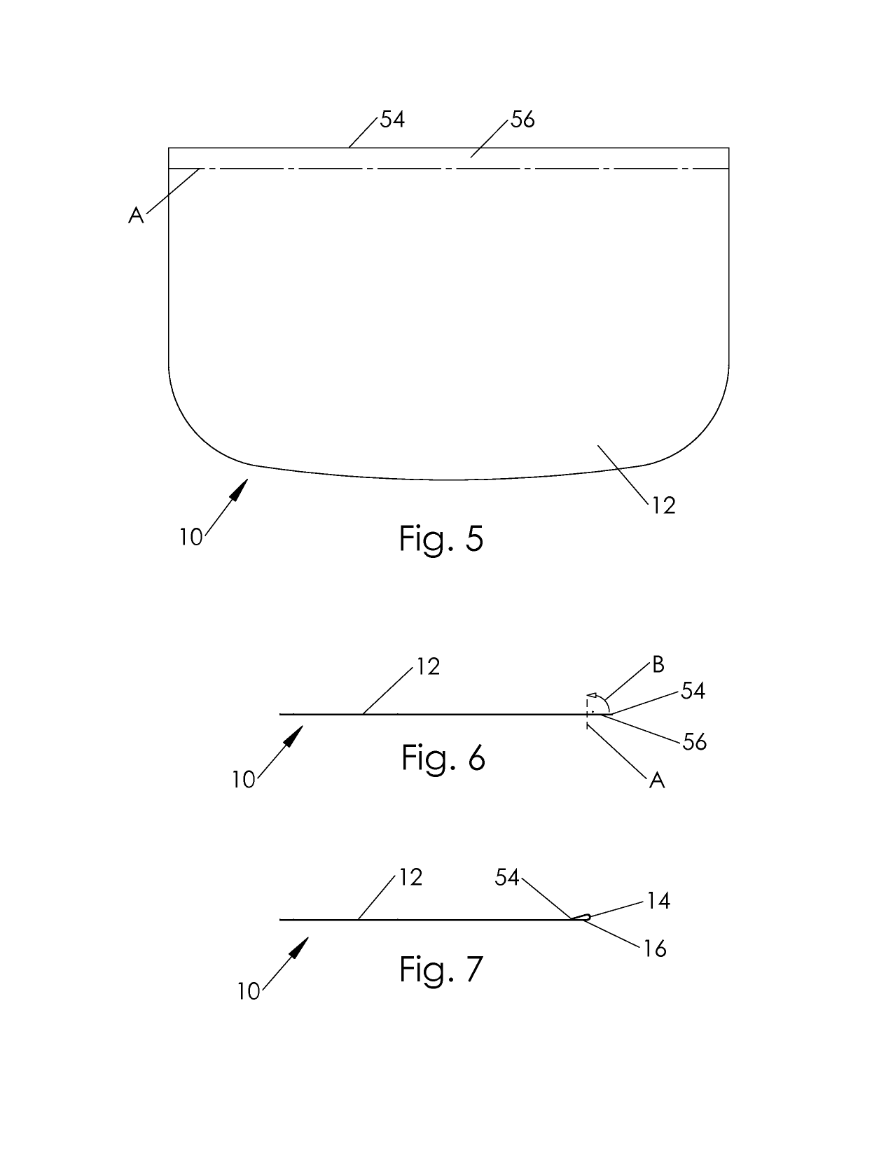

Manufacture of a sunshade 10 according to the first embodiment can be better understood with reference to FIGS. 5-7 which show respectively: a plan view of the first embodiment of the fabric sunshade as a blank during the process of manufacturing the sunshade; a schematic view of the first embodiment of the fabric sunshade from the side during the manufacturing process; and a schematic side view of the first embodiment of the completed fabric sunshade. In this first embodiment a single passageway 16 extends along the edge 14 of the sunshade 10. An edge portion 56 of the piece of fabric 12 is located adjacent an edge 54 of a fabric blank. The single passageway 16 comprises a folded over edge portion 56 of the piece of fabric that is fixed to the piece of fabric to form the passageway. That is to say, the edge portion 56 is folded about a fold line A in a direction B and then fixed to a main portion of the piece of fabric by stitching, an adhesive, heat staking or any other suitable means to form the passageway 16 which is open at both ends to accommodate the temples of a pair of glasses. Elastic characteristics of the four-way stretch fabric causes the fabric to grip the temples and aid in securing the sunshade to the glasses.

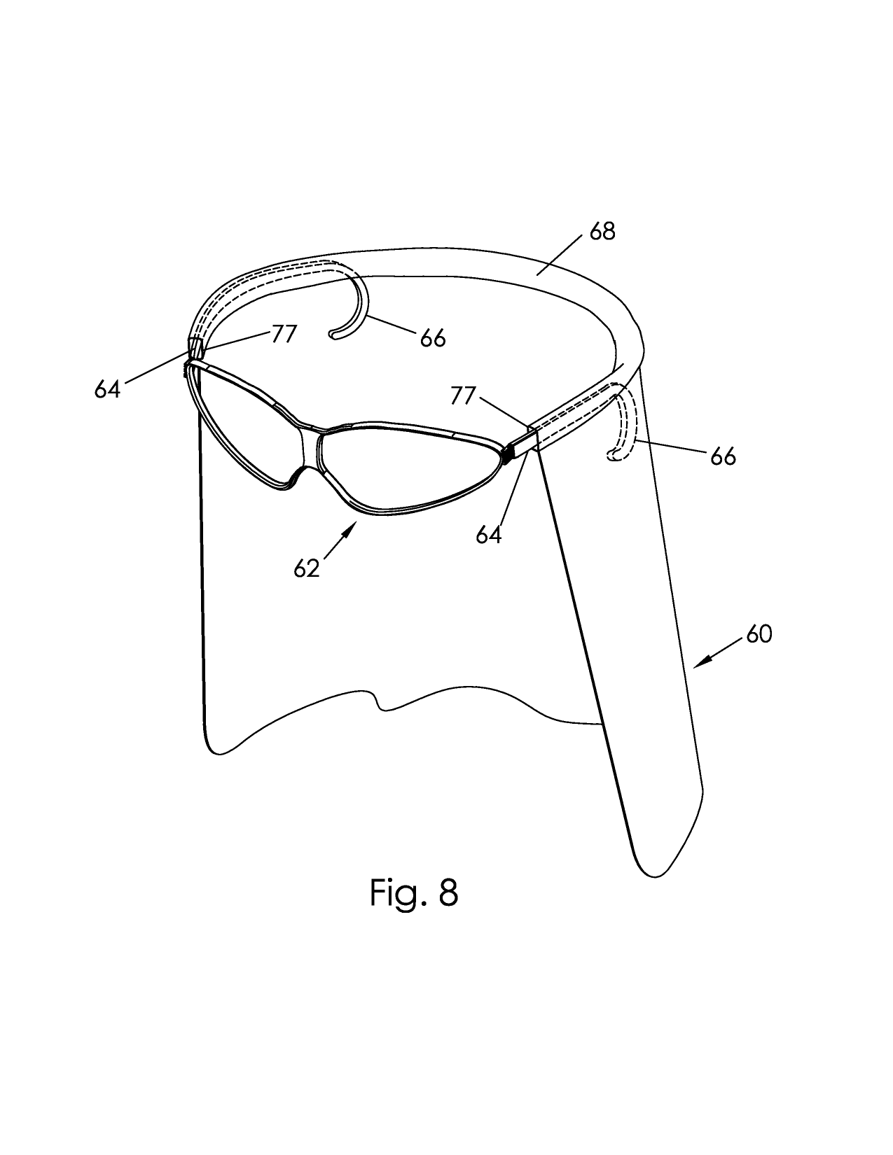

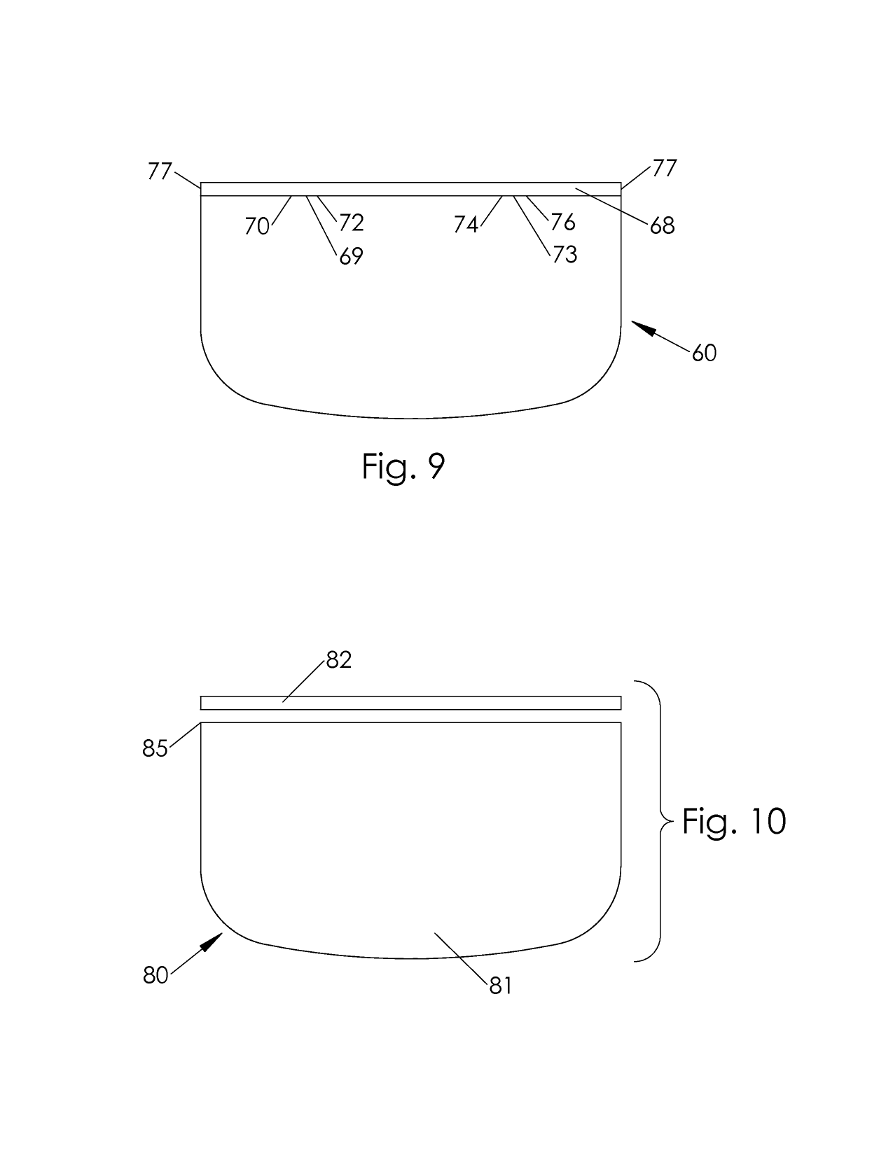

FIG. 8 is a perspective view of a second embodiment of a fabric sunshade 60 assembled with a pair of glasses 62, and FIG. 9 is a front plan view of the sunshade 60 of the second embodiment. The glasses 62 shown with this second embodiment differ from the glasses 30 shown with the first embodiment because the temples 64 in FIG. 8 have curved portions 66 that are intended to fit behind the ears of a wearer. A sunshade of the second embodiment is substantially the same as a sunshade of the first embodiment described above, however in this second embodiment the single passageway 68 has a pair of spaced apart slits 69, 73 therein extending therethrough disposed between ends 70, 72; 74, 76 of the slits for receiving the temples 64 of the glasses, each of the slits 69, 73 being spaced apart from the open ends 77 of the passageway 68. The slits are located at an intersection of the folded over portion of the piece of cloth with the main portion of the piece of cloth, and may be constructed by leaving gaps in a sewn seam, or adhesive, or heat staking that secures the folded over portion to the main portion of the piece of cloth.

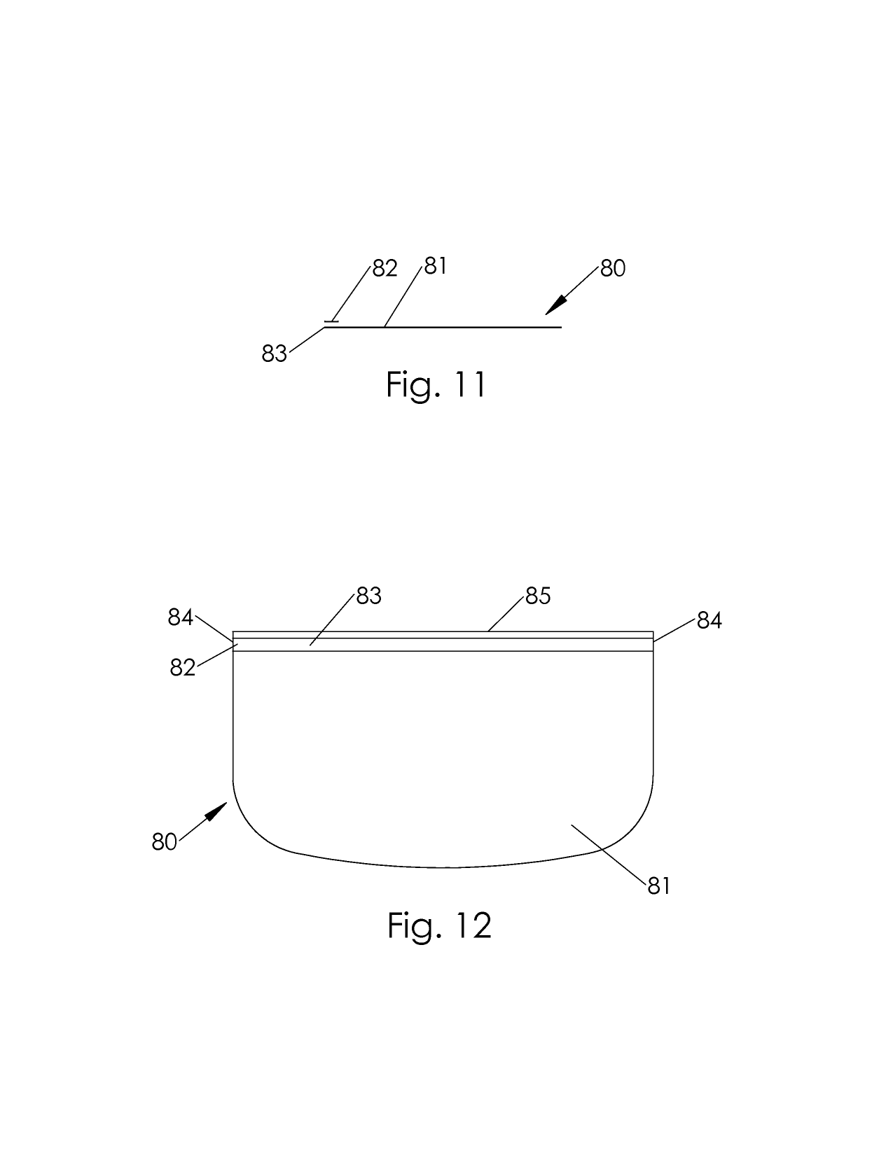

FIG. 10 is an exploded view of a third embodiment of a fabric sunshade 80 for use with glasses, and FIG. 11 is a schematic side view of the third embodiment of the fabric sunshade. The third embodiment begins with a fabric blank 81 which is a piece of cloth shaped like that shown in FIG. 5 with respect to the first embodiment. Instead of forming a passageway by folding over an edge portion of the fabric blank a strip of cloth 82 is fixed to the piece of cloth by sewing, an adhesive, heat staking or any other suitable means. As used herein and in the claims the term “strip” is understood to have its' common meaning of a piece of something such as cloth or paper that is much longer than it is wide. A front plan view of a sunshade of this third embodiment is shown in FIG. 12, which looks like the first embodiment as shown in a plan view in FIG. 3. That is to say, as shown best in FIGS. 11 and 12 a single passageway 83 is defined in part by a strip of fabric 82 attached to a piece of fabric 81. While the strip of fabric 82 may comprise any suitable fabric, it is preferably a four-way stretch fabric, and most preferably an elastic fabric. As used herein and in the claims an “elastic fabric” is understood to be a fabric containing interwoven or knit strands of flexible rubber or a similar substance allowing it to stretch and return to its original shape. It is understood that this third embodiment may include the feature of the second embodiment whereby the single passageway defined in part by the separate strip may have slits therein as shown in FIGS. 8 an 9 and described with respect thereto.

There may be provided in accordance with this third embodiment of the invention a sunshade comprising a piece of a four-way stretch fabric 81, the sunshade having an edge 85 and provided with a passageway 83 having a pair of open ends 84, the passageway located adjacent or near the edge 85 of the sunshade and extending at least substantially parallel to the edge of the sunshade for receiving the temples of a pair of glasses to attach the sunshade to the glasses, the passageway being formed at least in part by a strip 82 of an elastic fabric attached to the piece of four-way stretch fabric. When assembled with glasses the sunshade extends between the temples of the glasses and when the glasses and sunshade are worn by a person a portion of the passageway is disposed on a back side of the head of the person and the sunshade extends downward to shade at least a portion of a neck of the person.

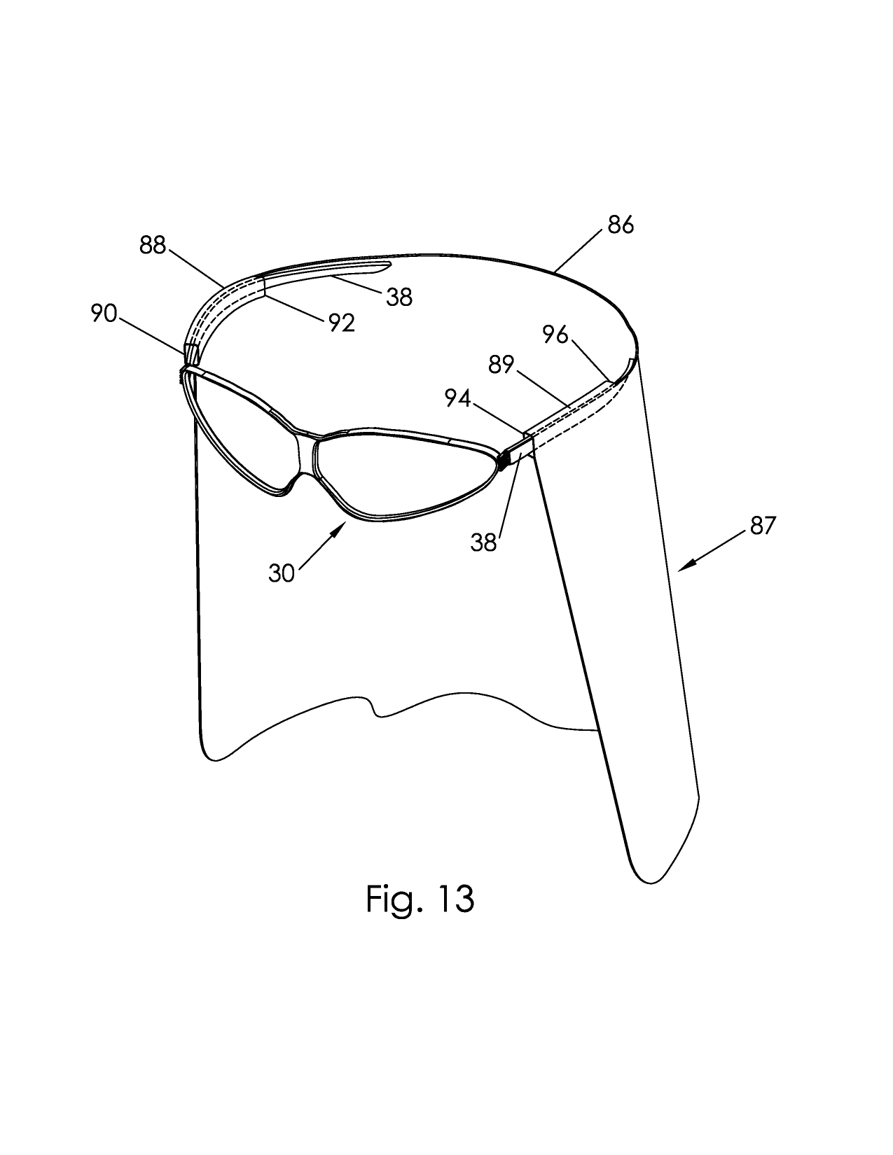

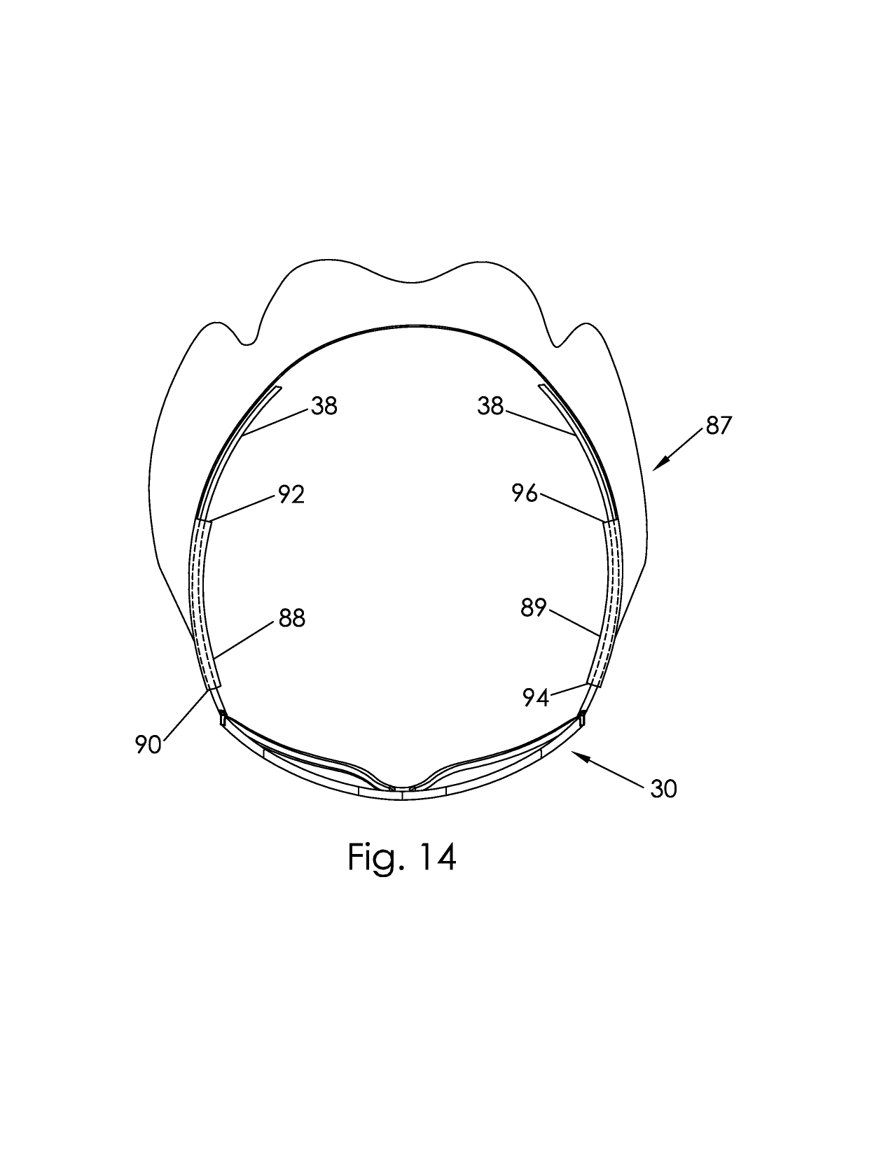

Referring next to FIGS. 13 and 14 there is shown a perspective view of a fourth embodiment of a fabric sunshade 87 assembled with a pair of glasses 10 and a top view looking down on the same assembly. This embodiment is substantially like the first embodiment described above with the exception that the fabric sunshade 87 has a pair of spaced apart passageways 88, 89 that are each located at or near an edge 86 of the sunshade and each have a pair of open ends 90, 92; 94, 96 wherein each of the passageways 82, 84 is configured to receive one of the temples 38 of the glasses 30.

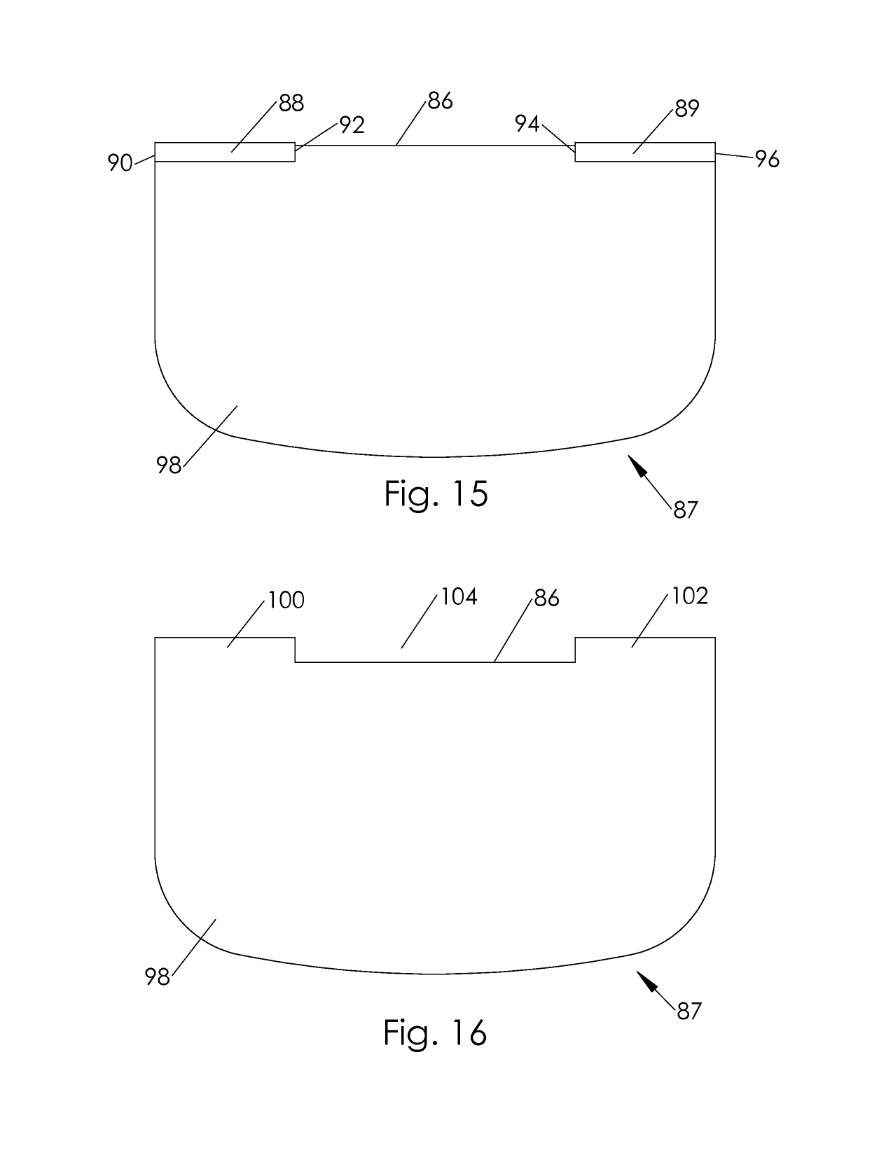

FIG. 15 is a front plan view of the fourth embodiment of the fabric sunshade 87, and FIG. 16 is a plan view of a piece of fabric 98 that is a blank used in the process of manufacturing the fourth embodiment. The piece of fabric has a pair of spaced apart edge portions 100, 102, in the form of tabs, spaced apart by an indentation 104. In this embodiment each of the passageways 88, 89 comprises a folded over edge portion 100, 102 of the piece of fabric 98 that is fixed to the piece of fabric to form a passageway. As described above with respect to the second embodiment referring to FIGS. 5-7 the edge portions 100, 102 are each folded about a fold line A in a direction B and then fixed to a main portion of the piece of fabric by stitching, an adhesive, heat staking or any other suitable means to form the passageways 88, 89 which each are open at both ends to accommodate the temples of a pair of glasses. Elastic characteristics of the four-way stretch fabric causes the fabric to grip the temples and aid in securing the sunshade to the glasses.

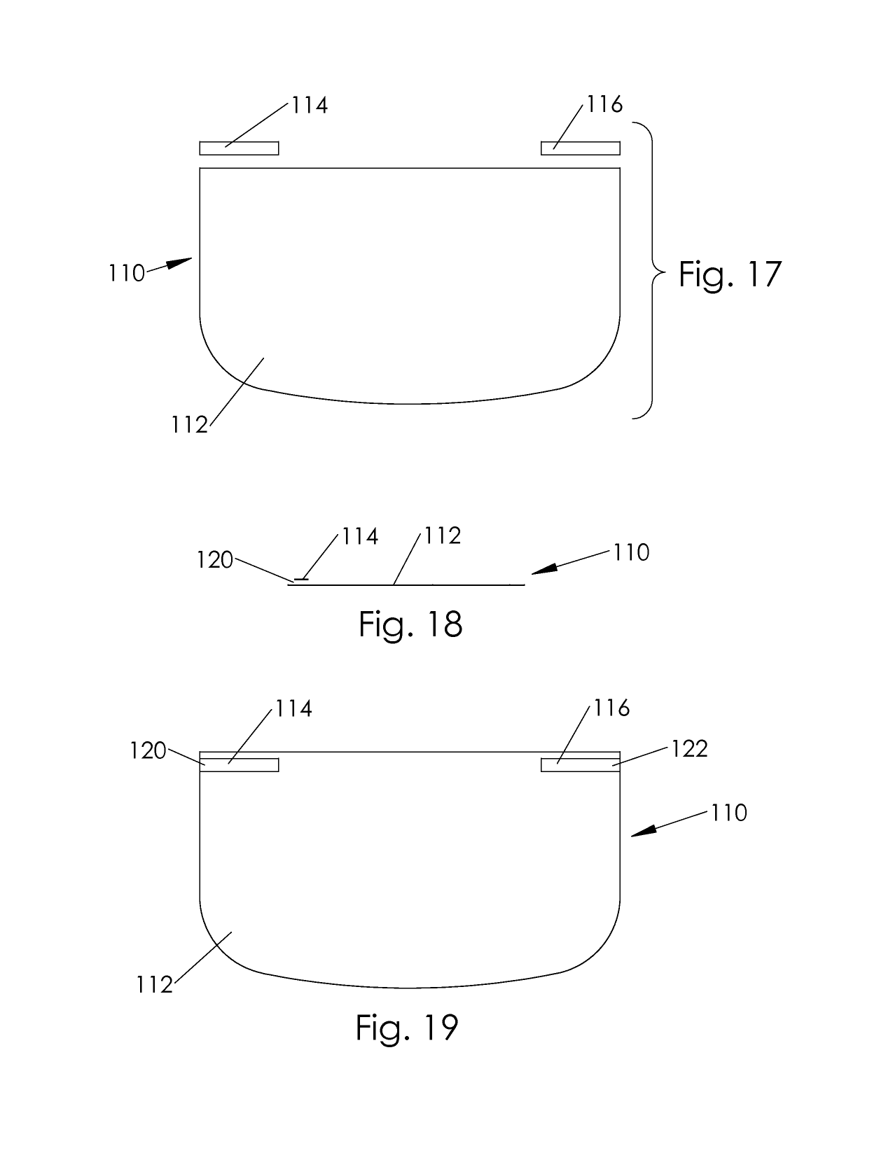

FIG. 17 is an exploded view of a fifth embodiment of a fabric sunshade 110 for use with glasses, FIG. 18 is a schematic side view of the fifth embodiment of the fabric sunshade, and FIG. 19 is a front plan view of the fifth embodiment. The fifth embodiment begins with a fabric blank 112 which is a piece of cloth shaped like that shown in FIG. 5 with respect to the first embodiment. Instead of forming a passageway by folding over an edge portion of the fabric blank two strips of cloth 114, 116 are fixed to the piece of cloth by sewing, an adhesive, heat staking or any other suitable means. That is to say the fifth embodiment is provided with a pair of spaced apart passageways 120, 122 wherein each of the passageways is defined in part by a strip of fabric 114, 116 attached to the piece of fabric 112. Each of the passageways has a pair of open ends for receiving the temples of a pair of glasses. While the strips of fabric 114, 116 may comprise any suitable fabric, it is preferably a four-way stretch fabric. When a fabric sunshade of the fifth embodiment is attached to a pair of glasses the assembly has substantially the structure shown in FIGS. 13 and 14.

While fabric sunshades according to any of the disclosed embodiments may be marketed as individual items, it is contemplated that a kit may be marketed comprising a pair of glasses, such as glasses with tinted lenses often called sunglasses, and a cloth sunshade that may be readily assembled with and separated from the glasses. This ease of attachment to and separation from glasses is an important advantage of the present invention because a person may wish to wear the same glasses for instance while driving an enclosed vehicle, then with a cloth sunshade when outside the vehicle and exposed to sunlight.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense. It is also to be understood that the following claims are intended to cover all of the generic and specific features of the invention herein described, and all statements of the scope of the invention which, as a matter of language, might be said to fall there between.

1. A sunshade for assembly with glasses having temples, the sunshade comprising a piece of fabric and the sunshade is provided with a single passageway located at or near a straight edge of the sunshade and extends along the straight edge of the sunshade, the single passageway having a pair of open ends and, a pair of spaced apart slits in a wall of the single passageway, each of the slits being spaced apart from the open ends of the single passageway, whereby the glasses may be attached to the sunshade by passing each of the temples of the glasses through an open end of the single passageway into the passageway and through a next adjacent slit in the wall of the single passageway such that the sunshade extends between the temples of the glasses and when the glasses and sunshade are worn by a person the sunshade extends downward to shade at least a portion of a neck of the person.

2. The sunshade of claim 1 wherein the fabric is a four-way stretch fabric.

3. (canceled)

4. (canceled)

5. The sunshade of claim 1 wherein the single passageway comprises a folded over edge portion of the piece of fabric that is fixed to the piece of fabric to form the passageway.

6. The sunshade of claim 2 wherein the single passageway comprises a folded over edge portion of the piece of fabric that is fixed to the piece of fabric to form the passageway.

7. (canceled)

8. (canceled)

9. A sunshade for assembly with glasses having temples, the sunshade comprising a piece of fabric and the sunshade is provided with a pair of spaced apart passageways that are each located at or near a straight edge of the sunshade and each passageway has a pair of open ends, whereby the glasses may be attached to the sunshade by passing each of the temples of the glasses through an open end of one of the passageways, through the passageway, and out of the other open end of the passageway such that the sunshade extends between the temples of the glasses and when the glasses and sunshade are worn by a person the sunshade extends downward to shade at least a portion of a neck of the person.

10. The sunshade of claim 9 wherein the fabric is a four-way stretch fabric.

11. The sunshade of claim 10 wherein each of the passageways comprises a folded over edge portion of the piece of fabric that is fixed to the piece of fabric to form a passageway.

12. The sunshade of claim 9 wherein each of the passageways comprises a folded over edge portion of the piece of fabric that is fixed to the piece of fabric to form a passageway.

13. The sunshade of claim 1 wherein the single passageway is defined in part by a strip of fabric attached to the piece of fabric.

14. The sunshade of claim 2 wherein the single passageway is defined in part by a strip of fabric attached to the piece of fabric.

15. The sunshade of claim 13 wherein the strip of fabric comprises an elastic fabric.

16. The sunshade of claim 14 wherein the strip of fabric comprises an elastic fabric.

17. (canceled)

18. (canceled)

19. (canceled)

20. A kit for shading at least a portion of a person's neck comprising:
(a) a pair of glasses having lenses that are dark at least when the lenses are exposed to sunlight and a pair of temples; and
(b) a sunshade comprising a piece of fabric, the sunshade having a straight edge and being provided with a pair of spaced apart passageways that are each located adjacent or near the straight edge of the sunshade and extending at least substantially parallel to the edge of the sunshade, each of the passageways having a pair of open ends whereby the glasses may be attached to the sunshade by passing each of the temples of the glasses through an open end of one of the passageways, through the passageway, and out of the other open end of the passageway such that the sunshade extends between the temples of the glasses and when the glasses and sunshade are worn by a person the sunshade extends downward to shade at least a portion of a neck of the person.

21. The sunshade of claim 9 wherein each of the passageways is defined in part by a strip of fabric attached to the piece of fabric.

22. The sunshade of claim 10 wherein each of the passageways is defined in part by a strip of fabric attached to the piece of fabric.
